(19) United States
(12) Patent Application Publication
(10) Pub. No.: US 2023/0360738 A1
(43) Pub. Date: Nov. 9, 2023

(54) Molecule Selection Using Simulation and Ranking Based on Binding Matrices

(71) Applicant: Massachusetts Institute of Technology, Cambridge, MA (US)

(72) Inventors: Rafael Gomez-Bombarelli, Somerville, MA (US); Daniel Schwalbe-Koda, Allston, MA (US)

(21) Appl. No.: 18/095,707

(22) Filed: Jan. 11, 2023

Related U.S. Application Data
(60) Provisional application No. 63/338,620, filed on May 5, 2022.

(51) Int. Cl. G16C 20/10; G06F 30/20

(52) U.S. Cl. CPC G16C 20/10; G06F 30/20

(57) ABSTRACT

A computer-implemented method calculates the affinity and templating ability of a molecule for a nanoporous host framework using a multi-factor index that uses simulation outcomes as component indices. Component factors of the multi-factor index may include, for example, binding energy, competition energy, and/or directivity energy. The multi-factor index may be used to analyze how known molecules template the formation of known frameworks.

### CROSS-REFERENCE TO RELATED APPLICATIONS

This application claims priority to U.S. Prov. Pat. App. No. 63/338,620, filed on May 5, 2022, entitled, “Molecule Selection Using Simulation and Ranking Based on Binding Matrices.”

### BACKGROUND

Zeolites are a class of nanoporous crystalline inorganic materials that have broad applications in the chemical industry. They are excellent catalytic materials thanks to their monodisperse pore size, structural variety that can shape-match a wide variety of substrates, tunable active sites through functionalization with acidic or metallic sites, easy and scalable synthesis from affordable starting materials, and robustness in operation.

Research in zeolite synthesis requires intensive trial and error because of the complex high-dimensional inputs that determine the synthetic outcome (framework, composition, crystal size) and catalytic ability from synthesis conditions and templating agents. About 250 different zeolites have been reported, of which only 10% are used commercially, but thousands are predicted to be theoretically possible. The complex interplay between synthesis conditions (temperature, pH, precursor composition, time, etc.), the use of crystalline seeds and templating ability of inorganic and organic structure directing agents (OSDA) are too complex for a traditional Edisonian approach.

### SUMMARY

A computer-implemented method performs the following steps. For each of a plurality of proposed molecules M, and for each of a plurality of proposed nanoporous host frameworks F, the method:
quantifies the interaction of the molecule M and the framework F with a physics-based simulation;
calculates, based on the simulation, for each of a plurality of component indices, a corresponding component index value associated with the molecule M:framework F pair; and
calculates a value of a multi-factor index associated with the molecule M:framework F pair based on the plurality of component index values associated with the molecule M:framework F pair, wherein the value of the multi-factor index represents the affinity of the molecule M to the framework F.

The method thereby generates a binding matrix containing the multi-factor index value for each molecule-framework pair. The method ranks the molecules M based on the multi-factor index values associated with the molecules M, and selects a subset of the molecules M based on the rankings.

Other features and advantages of various aspects and embodiments of the present invention will become apparent from the following description and from the claims.

### BRIEF DESCRIPTION OF THE DRAWINGS

FIG. 1 is a dataflow diagram of a system for selecting a subset of a plurality of proposed molecules based on a binding matrix according to one embodiment of the present invention.

FIG. 2 is a flowchart of a method performed by the system of FIG. 1 according to one embodiment of the present invention.

### DETAILED DESCRIPTION

Embodiments of the present invention include computer-implemented systems and methods for calculating the affinity and templating ability of a molecule for a nanoporous host framework using a multi-factor index that uses simulation outcomes as component indices. Until now, a single-factor index has been used: the binding energy of the molecule and the framework calculated as

E_{bind}=Eₚₒₛₑ−E_{molecule}−E_{framework},
where E_{bind} is the binding energy between templating agent and crystal, Eₚₒₛₑ is the energy of the bound pose, E_{molecule} is the energy of the isolated molecule (or the energy of all of the isolated molecules) and E_{framework} is the energy of the isolated framework. Energies can be calculated following a number of different protocols are methods (molecular dynamics, density functional theory), calculation schemes (constant pressure, constant volume, or others) and normalization schemes (normalized by number of atoms in the framework or number of OSDAs). In the past, a single-factor index (E_{bind}) has been used to rank molecules for prioritizing experiments and to discover templating materials for industrially relevant zeolites. Mostly, binding energies of different molecules towards a single framework have been used as one of the only design parameters when selecting an OSDA for synthesis of a given zeolite.

The inventors of the claimed subject matter herein have found that a multi-factor index, composed from a combination (e.g., a weighted average or geometric mean) of simpler indices, is more predictive of experimental outcomes than the binding energy by itself. Some components factors that can be included in the multi-factor index are, in any combination:
Binding energy (E_{bind})/normalized by framework atom or normalized by count of template molecules.

$${E}_{i j} = \frac{{E}_{b}}{{SiO}_{2}} or \frac{{E}_{b}}{OSDA}$$

Competition energy, which quantifies the templating ability of a given OSDA towards a zeolite when all other zeolite polymorphs are taken into consideration. It is defined by weighting exponentially E_{bind} of a given zeolite and molecule divided by the sum of such exponential weighting for all zeolites for that molecule.

$${C}_{i j} = \frac{exp ( - \frac{{E}_{i j}}{k T} )}{{Σ}_{j = zeo} exp ( - \frac{{E}_{i j}}{k T} )}$$

Directivity energy, which ranks the templating abilities of each molecule towards a framework when all OSDAs are taken into consideration.

$${D}_{i j} = \frac{exp ( - \frac{{E}_{i j}}{k T} )}{{Σ}_{i = OSDA} exp ( - \frac{{E}_{i j}}{k T} )}$$

The combination (e.g., weighted average or geometric mean) of all the scores gives the multi-factor index.

Embodiments of the present invention have been used to analyze how known molecules template the formation of known frameworks. We find that it is superior in predicting templating ability to the traditional single-factor index of binding energy. It is more effective at ranking which known molecules template known materials.

Embodiments of the present invention have also been used to identify templating agents for desired frameworks with high catalytic activity that have been realized in the lab, including both pure frameworks and intergrown frameworks.

Having described various embodiments of the present invention at a high degree of generality, certain embodiments of the present invention will now be described in more detail.

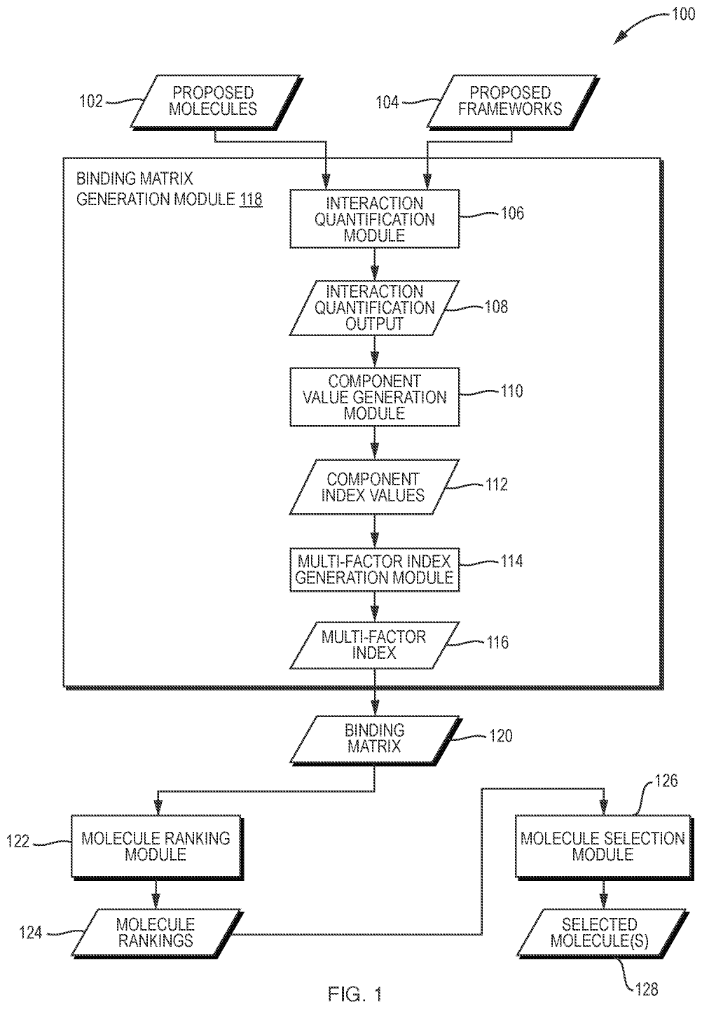

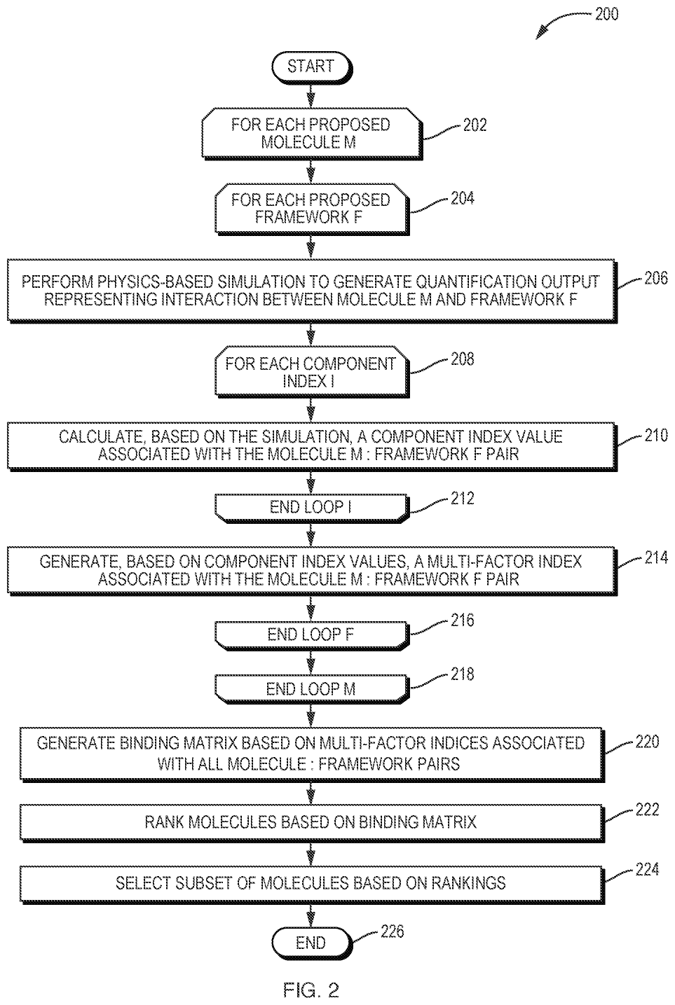

Referring to FIG. 1, a dataflow diagram is shown of a system 100 for selecting a subset of a plurality of proposed molecules based on a binding matrix according to one embodiment of the present invention. Referring to FIG. 2, a flowchart is shown of a method 200 performed by the system 100 of FIG. 1 according to one embodiment of the present invention.

The system 100 includes data 102 representing a plurality of proposed molecules. For ease of explanation, the data 102 will also be referred to herein simply as “the proposed molecules.” In practice, the proposed molecule data 102 may include any data representing a plurality of proposed molecules, such as in any of a variety of ways that are well-known to those having ordinary skill in the art. Some or all of the proposed molecules 102 may be organic structure directing agents (OSDAs). The term “templating agent” is used synonymously with OSDA herein. Some or all of the proposed molecules 102 may be known molecules. Some or all of the proposed molecules 102 may be hypothetical, and not yet realized, molecules.

The system 100 also includes data 104 representing a plurality of proposed nanoporous host frameworks. For ease of explanation, the data 104 will also be referred to herein simply as “the proposed frameworks.” In practice, the proposed framework data 104 may include any data representing a plurality of proposed nanoporous host frameworks, such as in any of a variety of ways that are well-known to those having ordinary skill in the art. Some or all of the proposed frameworks 104 may be known frameworks. Some or all of the proposed frameworks 104 may be hypothetical, and not yet realized, frameworks.

In general, a framework is a structure into which the atoms of a zeolite material can arrange themselves. Each zeolite belongs to one and only one framework, unless that zeolite is an intergrown zeolite, in which case multiple distinct frameworks are interwoven in the zeolite. The framework characterizes the geometric arrangement of the atoms, and may be any of a number of compositions (e.g., Si, Al, P, B, Ti, in different proportions). All the atoms in a framework are arranged as a network of tetrahedra connected through the edges. Some or all of the proposed frameworks 104 may be all-silica.

A zeolite implies a certain framework or set of frameworks (e.g., CHA, AEI), a certain composition (ratios of Si—Al and other elements), and a certain ordering of the various elements within the framework(s) (e.g., Si—Si—Al is different from Si—Al—Si in a hypothetical “chain-like” framework, even if both are 3-long and both have Si—Al 2:1). An OSDA will direct towards a certain framework in general, but may also show preference towards certain compositions or even atomic placements in the framework, given the same composition. So, in this sense, an OSDA may template both a framework and a zeolite. The system 100 and method 200 may, for example, address both sequentially, first finding the framework that is a best fit (regardless of composition) and then exploring the particular composition or ordering of various elements. If an OSDA directs towards two different frameworks with approximately equal affinity, the system 100 and method 200 may also address the possibility of intergrown zeolites.

The method 200 enters a loop over each of the proposed molecules 102 (FIG. 2, operation 202). The molecule that is the object of the current iteration of the loop entered into in operation 202 is referred to herein as “the current molecule” or “the molecule M.”

Within the loop entered into in operation 202, the method 200 enters a loop over each of the proposed frameworks 104 (FIG. 2, operation 204). The framework that is the object of the current iteration of the loop entered into in operation 204 is referred to herein as “the current framework” or “the framework F.”

The system 100 also includes an interaction quantification module 106, which performs a physics-based simulation of interactions between the molecule M and the framework F, and produces as output, based on the simulation, an interaction quantification output which quantifies one or more interactions between the module M and the framework F (FIG. 2, operation 206). Some or all of the proposed frameworks 104 may include at least one aluminum atom at a particular position, and quantifying the interaction(s) of the molecule M and the framework F in operation 206 may take into account the particular position.

The method 200 enters a loop over each of a plurality of component indices (FIG. 2, operation 208). The plurality of component indices may include, for example, some or all of the component indices disclosed herein. For example, the plurality of component indices may include any one or more of the following, in any combination, a binding energy index, a competition energy index, and a directivity energy index.

The system 100 also includes a component index value generation module 110 which calculates, based on the simulation, a component index value associated with the molecule M:framework F pair (FIG. 2, operation 210). The method 200 repeats operation 210 for all of the component indices (FIG. 2, operation 212), thereby generating a plurality of component index values 112. The component index value generation module 110 thereby generates, based on the simulation, for each of the plurality of component indices, a corresponding component index value associated with a distinct molecule M:framework F pair.

The system 100 also includes a multi-factor index generation module 114, which receives the component index values 112 as input, and which generates, based on the component index values 112, a multi-factor index 116 associated with the molecule M:framework F pair based on the plurality of component index values 112 associated with the molecule M:framework F pair, wherein the value of the multi-factor index represents an affinity of the molecule M to the framework F (FIG. 2, operation 214).

The interaction quantification output 108, component index values 112, and multi-factor index 116 described above are all generated for a single molecule M:framework F pair. The method 200 repeats operations 206-214 for each of the remaining proposed frameworks 104 (FIG. 2, operation 216), and repeats operations 204-216 for each of the remaining proposed molecules 102 (FIG. 2, operation 218), thereby generating additional instances of the interaction quantification output 108, component index values 112, and multi-factor index 116 for each molecule M:framework F (i.e., for each combination of proposed molecules 102 and proposed frameworks 104). In particular, operations 202-218 produce an instance of the multi-factor index 116 for each combination of proposed molecules 102 and proposed frameworks 104.

The interaction quantification module 106, component index value generation module 110, and multi-factor index generation module 114, are all part of a binding matrix generation module 118, which generates an instance of the multi-factor index 116 for each combination of the proposed molecules 102 and the proposed frameworks 104, in the manner disclosed above.

The binding matrix generation module 118 generates, based on the instances of the multi-factor index 116 corresponding to the combinations of the proposed molecules 102 and the proposed frameworks 104, a binding matrix 120, which contains, for each molecule-factor pair, the corresponding instance of the multi-factor index 116 (FIG. 2, operation 220).

The system 100 also includes a molecule ranking module 122, which receives the binding matrix 120 as input, and which ranks the molecules 102 based on the multi-factor index values associated with the molecules, thereby producing molecule rankings 124 as output (FIG. 2, operation 222).

The system 100 also includes a molecule selection module 126, which receives the molecule rankings 124 as input, and which selects a subset 128 of the molecules 102 based on the molecule rankings 124 (FIG. 2, operation 224). The molecule selection module 126 may select the subset 128 in any of a variety of ways, such as by selecting the N highest-rank molecules (where N may be selected to be any number), or by selecting the molecules in the top N % of the molecule rankings 124 (where N may be selected to be any number).

The set of proposed molecules 102 may change over time, and the method 200 of FIG. 2 may be performed on the revised set of proposed molecules 102. For example, one or more additional proposed molecules (i.e., proposed molecules which were not in the original set of proposed molecules 102) may be selected and added to the set of proposed molecules 102, thereby producing a revised set of proposed molecules (not shown). As another example, one or more molecules in the original set of proposed molecules 102 may be removed from the original set of proposed molecules 102, thereby producing a revised set of proposed molecules (not shown).

Regardless of how the revised set of proposed molecules is generated, the method 200 of FIG. 2 may be performed on the revised set of proposed molecules to produce a revised binding matrix (not shown) and a revised set of selected molecules (not shown). Such revisions to the set of proposed molecules may be made any number of times in any combination, and the method 200 of FIG. 2 may be performed on some or all of the revised sets of proposed molecules.

In some embodiments, the techniques described herein relate to a method performed by at least one computer processor executing computer program code stored on at least one non-transitory computer-readable medium, the method including: for each of a plurality of proposed molecules M: for each of a plurality of proposed nanoporous host frameworks F: quantifying an interaction of the molecule M and the framework F with a physics-based simulation; calculating, based on the simulation, for each of a plurality of component indices, a corresponding component index value associated with the molecule M:framework F pair; and calculating a value of a multi-factor index associated with the molecule M:framework F pair based on the plurality of component index values associated with the molecule M:framework F pair, wherein the value of the multi-factor index represents an affinity of the molecule M to the framework F; thereby generating a binding matrix containing the multi-factor index value for each molecule-factor pair; ranking the molecules M based on the multi-factor index values associated with the molecules M; and selecting a subset of the molecules M based on the rankings.

Selecting the subset of the molecules M may include selecting some number of the highest-ranking molecules M.

The plurality of indices may include a binding energy index, and calculating the component index value E_{bind} corresponding to the binding energy index for the molecule M:framework F pair may include calculating: Ebind=Epose−Emolecule−Eframework, wherein Ebind is the binding energy between the templating agent and crystal, wherein Epose is the energy of the bound pose, wherein Emolecule is the energy of the isolated molecule M, and wherein Eframework is the energy of the isolated framework F. Calculating the component index value Ebind corresponding to the binding energy index for the molecule M:framework F pair may further include normalizing the binding energy by framework atom. Calculating the component index value Ebind corresponding to the binding energy index for the molecule M:framework F pair may further include normalizing the binding energy by count of template molecules.

In some embodiments, the techniques described herein relate to a method, wherein the plurality of indices includes a competition energy index, which quantifies the templating ability of a given molecule towards the framework F when all other polymorphs of the framework F are taken into consideration.

The competition energy may be calculated as:

$${C}_{i j} = \frac{exp ( - \frac{{E}_{i j}}{k T} )}{{Σ}_{j = zeo} exp ( - \frac{{E}_{i j}}{k T} )}$$

The plurality of indices may include a directivity energy index, which ranks the templating ability of each of the molecules M towards the framework F when all OSDAs are taken into consideration. The directivity energy index may be calculated as:

$${D}_{i j} = \frac{exp ( - \frac{{E}_{i j}}{k T} )}{{Σ}_{i = OSDA} exp ( - \frac{{E}_{i j}}{k T} )}$$

The plurality of proposed nanoporous host frameworks F may include a plurality of zeolites.

Calculating the value of the multi-factor index associated with the molecule M:framework F pair may include calculating a geometric mean of the plurality of component index values associated with the molecule M:framework F pair.

The method may further include: selecting a plurality of additional proposed molecules; adding the plurality of additional proposed molecules to the plurality of proposed molecules to produce a revised set of proposed molecules M;′ and performing the method on the revised set of proposed molecules M′.

The method may further include: selecting a subset of the plurality of proposed molecules M; removing the subset from the plurality of proposed molecules to produce a revised set of proposed molecules M;′ and performing the method on the revised set of proposed molecules M′.

Selecting the subset of the plurality of proposed molecules M may include selecting the subset of the plurality of proposed molecules M based on the ranking of the plurality of proposed molecules M.

At least one of the plurality of proposed frameworks F may be a known framework.

At least one of the plurality of proposed frameworks F may be a hypothetical, and not yet realized, framework.

At least one of the plurality of proposed molecules M may be a known molecule.

At least one of the plurality of proposed molecules M may be a hypothetical, and not yet realized, molecule.

Each of the plurality of frameworks F may be all-silica.

At least one of the plurality of frameworks F may include at least one aluminum atom at a particular position, and wherein quantifying the interaction of the molecule M and the framework F may take into account the particular position.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention simulate molecules and nanoporous host frameworks and evaluate the molecules based on those simulations. Such a function cannot be performed mentally or manually, and is inherently rooted in computer technology.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

Any step or act disclosed herein as being performed, or capable of being performed, by a computer or other machine, may be performed automatically by a computer or other machine, whether or not explicitly disclosed as such herein. A step or act that is performed automatically is performed solely by a computer or other machine, without human intervention. A step or act that is performed automatically may, for example, operate solely on inputs received from a computer or other machine, and not from a human. A step or act that is performed automatically may, for example, be initiated by a signal received from a computer or other machine, and not from a human. A step or act that is performed automatically may, for example, provide output to a computer or other machine, and not to a human.

The terms “A or B,” “at least one of A or/and B,” “at least one of A and B,” “at least one of A or B,” or “one or more of A or/and B” used in the various embodiments of the present disclosure include any and all combinations of words enumerated with it. For example, “A or B,” “at least one of A and B” or “at least one of A or B” may mean: (1) including at least one A, (2) including at least one B, (3) including either A or B, or (4) including both at least one A and at least one B.

What is claimed is:

1. A method performed by at least one computer processor executing computer program code stored on at least one non-transitory computer-readable medium, the method comprising:
for each of a plurality of proposed molecules M:
for each of a plurality of proposed nanoporous host frameworks F:
quantifying an interaction of the molecule M and the framework F with a physics-based simulation;
calculating, based on the simulation, for each of a plurality of component indices, a corresponding component index value associated with the molecule M:framework F pair; and
calculating a value of a multi-factor index associated with the molecule M:framework F pair based on the plurality of component index values associated with the molecule M:framework F pair, wherein the value of the multi-factor index represents an affinity of the molecule M to the framework F;
thereby generating a binding matrix containing the multi-factor index value for each molecule-factor pair;
ranking the molecules M based on the multi-factor index values associated with the molecules M; and
selecting a subset of the molecules M based on the rankings.

2. The method of claim 1, wherein selecting the subset of the molecules M comprises selecting some number of the highest-ranking molecules M.

3. The method of claim 1, wherein the plurality of indices comprises a binding energy index, and wherein calculating the component index value E_{bind} corresponding to the binding energy index for the molecule M:framework F pair comprises calculating:
E_{bind}=Eₚₒₛₑ−E_{molecule}−E_{framework},
wherein E_{bind} is the binding energy between the templating agent and crystal,
wherein Eₚₒₛₑ is the energy of the bound pose,
wherein E_{molecule} is the energy of the isolated molecule M, and
wherein E_{framework} is the energy of the isolated framework F.

4. The method of claim 3, wherein calculating the component index value E_{bind} corresponding to the binding energy index for the molecule M:framework F pair further comprises normalizing the binding energy by framework atom.

5. The method of claim 3, wherein calculating the component index value E_{bind} corresponding to the binding energy index for the molecule M:framework F pair further comprises normalizing the binding energy by count of template molecules.

6. The method of claim 1, wherein the plurality of indices comprises a competition energy index, which quantifies the templating ability of a given molecule towards the framework F when all other polymorphs of the framework F are taken into consideration.

7. The method of claim 6, wherein the competition energy is calculated as:
$${C}_{i j} = \frac{exp ( - \frac{{E}_{i j}}{k T} )}{{Σ}_{j = zeo} exp ( - \frac{{E}_{i j}}{k T} )}$$

8. The method of claim 1, wherein the plurality of indices comprises a directivity energy index, which ranks the templating ability of each of the molecules M towards the framework F when all OSDAs are taken into consideration.

9. The method of claim 8, wherein the directivity energy index is calculated as:
$${D}_{i j} = \frac{exp ( - \frac{{E}_{i j}}{k T} )}{{Σ}_{i = OSDA} exp ( - \frac{{E}_{i j}}{k T} )}$$

10. The method of claim 1, wherein the plurality of proposed nanoporous host frameworks F comprises a plurality of zeolites.

11. The method of claim 1, wherein calculating the value of the multi-factor index associated with the molecule M:framework F pair comprises calculating a geometric mean of the plurality of component index values associated with the molecule M:framework F pair.

12. The method of claim 1, further comprising:
selecting a plurality of additional proposed molecules;
adding the plurality of additional proposed molecules to the plurality of proposed molecules to produce a revised set of proposed molecules M;′ and
performing the method of claim 1 on the revised set of proposed molecules M′.

13. The method of claim 1, further comprising:
selecting a subset of the plurality of proposed molecules M;
removing the subset from the plurality of proposed molecules to produce a revised set of proposed molecules M;′ and
performing the method of claim 1 on the revised set of proposed molecules M′.

14. The method of claim 13, wherein selecting the subset of the plurality of proposed molecules M comprises selecting the subset of the plurality of proposed molecules M based on the ranking of the plurality of proposed molecules M.

15. The method of claim 1, wherein at least one of the plurality of proposed frameworks F is a known framework.

16. The method of claim 1, wherein at least one of the plurality of proposed frameworks F is a hypothetical, and not yet realized, framework.

17. The method of claim 1, wherein at least one of the plurality of proposed molecules M is a known molecule.

18. The method of claim 1, wherein at least one of the plurality of proposed molecules M is a hypothetical, and not yet realized, molecule.

19. The method of claim 1, wherein each of the plurality of frameworks F is all-silica.

20. The method of claim 1, wherein at least one of the plurality of frameworks F includes at least one aluminum atom at a particular position, and wherein quantifying the interaction of the molecule M and the framework F takes into account the particular position.

21. A system comprising at least one non-transitory computer-readable medium having computer program code stored thereon, wherein the computer program code is executable by at least one computer processor to perform a method, the method comprising:
for each of a plurality of proposed molecules M:
for each of a plurality of proposed nanoporous host frameworks F:
quantifying an interaction of the molecule M and the framework F with a physics-based simulation;
calculating, based on the simulation, for each of a plurality of component indices, a corresponding component index value associated with the molecule M:framework F pair; and
calculating a value of a multi-factor index associated with the molecule M:framework F pair based on the plurality of component index values associated with the molecule M:framework F pair, wherein the value of the multi-factor index represents an affinity of the molecule M to the framework F;
thereby generating a binding matrix containing the multi-factor index value for each molecule-factor pair;
ranking the molecules M based on the multi-factor index values associated with the molecules M; and
selecting a subset of the molecules M based on the rankings.

22. The system of claim 21, wherein selecting the subset of the molecules M comprises selecting some number of the highest-ranking molecules M.

23. The system of claim 21, wherein the plurality of indices comprises a binding energy index, and wherein calculating the component index value E_{bind} corresponding to the binding energy index for the molecule M:framework F pair comprises calculating:
E_{bind}=Eₚₒₛₑ−E_{molecule}−E_{framework}
wherein E_{bind} is the binding energy between the templating agent and crystal,
wherein Eₚₒₛₑ is the energy of the bound pose,
wherein E_{molecule} is the energy of the isolated molecule M, and
wherein E_{framework} is the energy of the isolated framework F.

24. The system of claim 23, wherein calculating the component index value E_{bind} corresponding to the binding energy index for the molecule M:framework F pair further comprises normalizing the binding energy by framework atom.

25. The system of claim 23, wherein calculating the component index value E_{bind} corresponding to the binding energy index for the molecule M:framework F pair further comprises normalizing the binding energy by count of template molecules.

26. The system of claim 21, wherein the plurality of indices comprises a competition energy index, which quantifies the templating ability of a given molecule towards the framework F when all other polymorphs of the framework F are taken into consideration.

27. The system of claim 26, wherein the competition energy is calculated as:
$${C}_{i j} = \frac{exp ( - \frac{{E}_{i j}}{k T} )}{{Σ}_{j = zeo} exp ( - \frac{{E}_{i j}}{k T} )}$$

28. The system of claim 21, wherein the plurality of indices comprises a directivity energy index, which ranks the templating ability of each of the molecules M towards the framework F when all OSDAs are taken into consideration.

29. The system of claim 28, wherein the directivity energy index is calculated as:
$${D}_{i j} = \frac{exp ( - \frac{{E}_{i j}}{k T} )}{{Σ}_{i = OSDA} exp ( - \frac{{E}_{i j}}{k T} )}$$

30. The method of claim 21, wherein the plurality of proposed nanoporous host frameworks F comprises a plurality of zeolites.

31. The system of claim 21, wherein calculating the value of the multi-factor index associated with the molecule M:framework F pair comprises calculating a geometric mean of the plurality of component index values associated with the molecule M:framework F pair.

32. The system of claim 21, wherein the method further comprises:
selecting a plurality of additional proposed molecules;
adding the plurality of additional proposed molecules to the plurality of proposed molecules to produce a revised set of proposed molecules M;′ and
performing the method of claim 1 on the revised set of proposed molecules M′.

33. The system of claim 21, wherein the method further comprises:
selecting a subset of the plurality of proposed molecules M;
removing the subset from the plurality of proposed molecules to produce a revised set of proposed molecules M;′ and
performing the method of claim 1 on the revised set of proposed molecules M′.

34. The system of claim 33, wherein selecting the subset of the plurality of proposed molecules M comprises selecting the subset of the plurality of proposed molecules M based on the ranking of the plurality of proposed molecules M.

35. The system of claim 21, wherein at least one of the plurality of proposed frameworks F is a known framework.

36. The system of claim 21, wherein at least one of the plurality of proposed frameworks F is a hypothetical, and not yet realized, framework.

37. The system of claim 21, wherein at least one of the plurality of proposed molecules M is a known molecule.

38. The system of claim 21, wherein at least one of the plurality of proposed molecules M is a hypothetical, and not yet realized, molecule.

39. The system of claim 21, wherein each of the plurality of frameworks F is all-silica.

40. The system of claim 21, wherein at least one of the plurality of frameworks F includes at least one aluminum atom at a particular position, and wherein quantifying the interaction of the molecule M and the framework F takes into account the particular position.
